(19) United States
(12) Patent Application Publication
(10) Pub. No.: US 2026/0179772 A1
(43) Pub. Date: Jun. 25, 2026

(54) PREDICTING HEALTH STATES OF CATS

(71) Applicant: Societe des Produits Nestle S.A., Vevey (CH)

(72) Inventors: Ragen Trudelle-Schwarz McGowan, O'Fallon, IL (US); Natalie Langenfeld-McCoy, Moro, IL (US); Helber Dussan, Saint Louis, MO (US); Olivia Arndt, Saint Joseph, MO (US); Nicholas Schoeneck, Saint Joseph, MO (US); Nikhil Kakarlapudi, Andhra Pradesh (IN); Devanand Rajeev, Kollam (IN); Sanjana Panda, Maharashtra (IN); Siddharth Jain, Madhya Pradesh (IN); Praveenkumar Ganeshkumar, Nadu (IN); Somil Rastogi, Haryan (IN)

(73) Assignee: Societe des Produits Nestle S.A., Vevey (CH)

(21) Appl. No.: 19/406,088

(22) Filed: Dec. 2, 2025

Related U.S. Application Data
(60) Provisional application No. 63/736,050, filed on Dec. 19, 2024.

(51) Int. Cl. G16H 50/20; A01K 1/01

(52) U.S. Cl. CPC G16H 50/20; A01K 1/0107; A01K 1/0114

(57) ABSTRACT

The present disclosure provides systems and methods for predicting the health state of cats, e.g., renal health. The method can include obtaining load data over time from interactions of a cat with a litter box, e.g., a smart litter box system, and using the load data in conjunction with a machine learning model with feature recognition of at least one feature based on the load data that indicates compromised health. The method can also include predicting the compromised renal health of the cat is with at least about 75% renal precision using at least a 0.5 probability threshold based on the feature recognition of the at least one feature.

### CROSS REFERENCE TO RELATED APPLICATION

This application claims priority to U.S. Provisional Application Ser. No. 63/736,050 filed Dec. 19, 2024 the disclosure of which is incorporated in its entirety herein by this reference.

### BACKGROUND

Cat behavior can often be reflective of physical or mental health conditions, but it can be difficult to recognize behavior patterns that may be useful in diagnosing specific diseases, particularly at an early stage. As an example, to establish a health state of a cat, e.g., a diagnosis of a health condition such as a renal, urinary, or digestive disorder, it is useful to quantify any of a number of clinical signs to establish the health state, which may include an appropriate diagnosis of a disease. Even with the help of a veterinarian, a common hurdle to correctly diagnosing these types of diseases relates to providing the veterinarian with accurate information about fluctuations in the elimination behavior of the cat. Typically, veterinarians have traditionally had to rely on the pet owner's direct observations, interpretations, and estimates regarding quantification of changes in elimination behavior, which may not be reliable. For example, cat owners often place litter boxes in secluded areas and/or some cat owners opt to use a covered box, making observations too sporadic to be meaningful. To complicate this, it is common for cats to exhibit secretive or nocturnal voiding behaviors. These and other issues can make it difficult for a veterinarian to obtain accurate observational information from the pet owner. Thus, it would be beneficial for cat owners to have a way to track various litter box interactions in a way that is reliable, and at the same time not disrupt the cat as it relates to the cat's interaction with the litter box. This type of information may be useful to a pet owner and/or veterinarian in making a proper diagnosis, for example.

### BRIEF DESCRIPTION OF THE FIGURES

FIG. 1A schematically illustrates example animal health monitoring systems, which can include a smart litter box system, in accordance with the present disclosure.

FIGS. 1B to 1D illustrate example smart litter box systems, which can include a litter box container, a platform (which may or may not be integrated with the litter box container), and a plurality of load cells, in accordance with the present disclosure.

FIG. 2 illustrates a conceptual overview of example events that may occur using the animal health monitoring systems with the smart litter box systems in accordance with the present disclosure.

FIG. 3 illustrates an example graph illustrating differences in load signal from individual load cells as a cat interacts with a smart litter box system in accordance with the present disclosure.

FIG. 4 illustrates an example graph notating various phases of interaction of a cat with a smart litter box system in accordance with the present disclosure.

FIGS. 5A-5G illustrate multiple graphs of various features generated by comparing populations of healthy cats and renal cats with respect to their interaction with smart litter box systems in accordance with the present disclosure.

### DETAILED DESCRIPTION

In accordance with examples of the present disclosure, through a detailed collection of data from a population of cats as it relates to their litter box activity as a function of time, machine learning algorithms have been developed that assist with identifying the current health state of a cat. This technology can be used in connection with smart litter box systems that can detect cat behavior based on its interactions with the litter box. More specifically, artificial intelligence or machine-learning models can be trained and validated to distinguish between the litter box behavior of healthy cats and unhealthy cats. For example, longitudinal data collected from a population of cats, some of which are healthy and some of which are known to have a disease or health condition, can be inputted into machine learning models, and features identified to be associated with compromised cats can be used to predict the health state of the cat. To illustrate using renal health as an example, data can be collected over multiple days, e.g., at least 3 days or about 7 days, and aggregate summaries can be developed using various parameters or features that may indicate the presence of the disease or health condition. In the case of renal health, features may include number of daily urinations, cat weight variability, number of visits to the litter box, time spent in the litter box, time spent covering eliminations, time spent finding locations, intensity of elimination covering, weight of the elimination, etc. Whether there is an increase or decrease in these features can indicate whether a cat is renal or healthy, for example. Renal health compromise features (including “increase” or “decrease” are provided hereinafter). Comparing these features between two groups of cats, e.g., healthy cats and renal compromised cats, a machine learning model can be generated that reasonably accurately predicts the health state of the cat as either healthy or compromised. For diseases other than those related to compromised renal health, other sets of features may be selected for use to be predictive of the health states of cats.

In addition to those above, features that may indicate the presence of the disease or health condition may further include any combination of: number of combined elimination events over a predetermined duration, number of defecation events over a predetermined duration, number of non elimination events over a predetermined duration, number of urination events over a predetermined duration, number of total visits over a predetermined duration, mean/median event duration of combined elimination events over a predetermined duration, mean/median event duration of defecation events over a predetermined duration, mean/median event duration of non elimination events over a predetermined duration, mean/median event duration of urination events over a predetermined duration, mean/median entry & digging duration of combined elimination events over a predetermined duration, mean/median entry & digging duration of defecation events over a predetermined duration, mean/median entry & digging duration of non elimination events over a predetermined duration, mean/median entry & digging duration of urination events over a predetermined duration, mean/median elimination duration of combined elimination events over a predetermined duration, mean/median elimination duration of defecation events over a predetermined duration, mean/median elimination duration of non elimination events over a predetermined duration, mean/median elimination duration of urination events over a predetermined duration, mean/median transition/covering duration of combined elimination events over a predetermined duration, mean/median transition/covering duration of defecation events over a predetermined duration, mean/median transition/covering duration of non elimination events over a predetermined duration, mean/median transition/covering duration of urination events over a predetermined duration, mean/median second elimination duration of combined elimination events over a predetermined duration, mean/median second elimination duration of defecation events over a predetermined duration, mean/median second elimination duration of non elimination events over a predetermined duration, mean/median second elimination duration of urination events over a predetermined duration, mean/median covering & exit duration of combined elimination events over a predetermined duration, mean/median covering & exit duration of defecation events over a predetermined duration, mean/median covering & exit duration of non elimination events over a predetermined duration, mean/median covering & exit duration of urination events over a predetermined duration, mean/median digging up intensity of combined elimination events over a predetermined duration, mean/median digging up intensity of defecation events over a predetermined duration, mean/median digging up intensity of non elimination events over a predetermined duration, mean/median digging up intensity of urination events over a predetermined duration, mean/median covering up intensity of combined elimination events over a predetermined duration, mean/median covering up intensity of defecation events over a predetermined duration, mean/median covering up intensity of non elimination events over a predetermined duration, mean/median covering up intensity of urination events over a predetermined duration, mean/median event duration over a predetermined duration, mean/median entry & digging duration over a predetermined duration, mean/median elimination duration over a predetermined duration, mean/median transition/covering duration over a predetermined duration, mean/median second elimination duration over a predetermined duration, mean/median covering & exit duration over a predetermined duration, mean/median digging up intensity over a predetermined duration, mean/median covering up intensity over a predetermined duration, mean/median number of entries per event for combined elimination events over a predetermined duration, mean/median number of entries per event for defecation events over a predetermined duration, mean/median number of entries per event for non elimination events over a predetermined duration, mean/median number of entries per event for urination events over a predetermined duration, mean/median number of entries per event over a predetermined duration, sum of weight of output of combined elimination events over a predetermined duration, sum of weight of output of defecation events over a predetermined duration, sum of weight of output of non elimination events over a predetermined duration, sum of weight of output of urination events over a predetermined duration, mean/median of weight of output of combined elimination events over a predetermined duration, mean/median of weight of output of defecation events over a predetermined duration, mean/median of weight of output of non elimination events over a predetermined duration, mean/median of weight of output of urination events over a predetermined duration, sum of weight of output over a predetermined duration, mean/median of weight of output over a predetermined duration, standard deviation of weight (g) over a predetermined duration, standard deviation of weight (oz) over a predetermined duration, increase in weight (oz) over a predetermined duration, increase in weight (oz) over a predetermined duration, decrease in weight (oz) over a predetermined duration, and decrease in weight (oz) over a predetermined duration.

This technology, in combination with the use of a smart litter box system, e.g., a litter box adapted to collect load data, can be used with a cat in a way that is non-invasive, e.g., no need for video cameras, smart collars, or other items that may disrupt cat behavior, and can provide enough animal behavior data for the machine learning algorithm to make physical and/or health predictions that are typically more accurate than that which would occur by simple behavior observation over time. Thus, the present disclosure includes technology, e.g., machine learning model(s) used in combination with a smart litter box system, that is capable of distinguishing between the litter box behavior of healthy cats and unhealthy cats, e.g., renal disease, urinary tract disease, arthritis, hyperthyroidism, chronic enteropathy, Feline idiopathic cystitis, diabetes, etc. To provide an initial example, a machine learning model can be trained to predict whether a cat is healthy or renal based on cat interaction with a smart litter box system equipped with multiple load sensors for independently collecting load data. In accordance with this, the present disclosure relates to the field of animal behavior monitoring, and more particularly, to machine learning algorithms or models used in combination with load data collected by smart litter box systems for predicting physical and/or health states of cats.

In some examples, a method of predicting renal health of a cat, under the control of at least one processor, can include obtaining load data over time from interactions of a cat with a smart litter box system, using the load data in conjunction with a machine learning model including at least one feature indicates compromised renal health, and predicting the compromised renal health of the cat. The smart litter box system can include or be associated with a plurality of load sensors that are separated from one another and independently receive changing pressure inputs from the smart litter box system resulting from the interactions. The compromised renal health can be based on a comparison with benchmark values, e.g., average values over multiple days, established from a population of healthy cats. The at least one feature can include an increased number of daily elimination events, or can include multiple features selected from increased variability of weight, increased number of daily urination events, similar duration of voiding phase of elimination events, decreased duration of finding/digging in litter, decreased duration of covering, decreased duration of entirety of events, decreased intensity of covering, decreased intensity of finding/digging, decreased number of defecation events, decreased weight of each elimination, and/or increased number of smart litter box system visits. For some health states of cats, there could be increases, such as an increased number of urinations, increased number of defecations, increased duration of events, etc. The compromised renal health of the cat can be with at least about 75% renal precision using at least a 0.5 probability threshold based on the feature recognition of the at least one feature. Combining increased daily elimination events with at least one of the other features can result in at least 80% renal precision. Combining increased daily elimination events with at least two of the other features can result in at least 85% renal precision. Increasing the probability threshold to at least about 0.7 can increase the renal precision to at least about 80%. Adding an overrule, such as a renal precision heuristic overrule based on a change in number of daily urinations, can further increase the renal precision.

In another example, an animal health monitoring system can include a smart litter box system associated with or including a plurality of load sensors to obtain load data. Individual load sensors of the plurality of load sensors can be separated from one another and independently receive pressure inputs as a result of cat interaction with the smart litter box system and litter contained therein. The system can further include a data communicator configured to communicate the load data from the plurality of load sensor, a processor, and a memory. The memory can store instructions that, when executed by the processor, receives the load data from the data communicator, uses the load data in conjunction with a machine learning model that includes feature recognition of at least one feature based on the load data, wherein the at least one feature indicates compromised health compared to benchmark averages established from a population of healthy cats, and predicts the compromised health of the cat is with at least about 60% precision (or at least about 70% or at least about 75% precision) using at least a 0.5 probability threshold (or from about 0.5 to about 0.9 precision, e.g., at least 0.7 probability threshold) based on feature recognition of the at least one feature (or at least two features or at least three features). The choice of features can be the same as that described with respect to the method of predicting renal health of a cat described above and elsewhere herein.

In another example, a method of predicting a health state of a cat, under the control of at least one processor can include obtaining load data over time from interactions of a cat with a smart litter box system, using the load data in conjunction with a machine learning model, the machine learning model including feature recognition of at least one feature based on the load data, and predicting the compromised health of the cat is with at least about 60% precision (or at least about 70% precision or at least about 75% precision) using at least a 0.5 probability threshold (or from about 0.5 to about 0.9 precision, e.g., at least 0.7 probability threshold) based on feature recognition of the at least one feature (or at least two features or at least three features). The smart litter box system can be associated with or include a plurality of load sensors that are separated from one another and independently receive changing pressure inputs from the smart litter box system resulting from the interactions. Furthermore, the at least one feature can indicate compromised health compared to benchmark values, e.g., average values of healthy cats, established from a population of healthy cats. In this example, predicting the unhealthy state includes predicting the presence of a health condition selected from: urinary tract disease with the at least one feature used for predicting the urinary tract disease includes increased total event duration, arthritis with the at least one feature used for predicting the arthritis includes decreased dig up duration, decreased total event duration, decreased dig up intensity, or decreased cover up intensity, hyperthyroidism with the at least one feature used for predicting the hyperthyroidism includes increased dig up duration, Feline idiopathic cystitis with the at least one feature used for predicting the Feline idiopathic cystitis includes increased number of non-elimination events, increased defecation output duration, or decrease in total even duration, or diabetes with the at least one feature used for predicting the diabetes includes increased urination weight output, decreased defecation weight of output, decreased number of defecation events.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### Predicting Renal Health of Cats

In accordance with the present disclosure, systems and methods for predicting the renal health (or other health issues) of cats can be based on data collected from a litter box where the cat typically eliminates. In examples of the present disclosure, the renal health prediction systems of the present disclosure may include the use of a smart litter box system that collects load data from multiple sensors. The load sensors can monitor the distribution of the weight of the animal, cat movement, and/or the time the animal is located within or in contact with the smart litter box system. For example, the load sensor data can be used to track a cat's movement patterns in the litter box, identify non-cat interactions with the box, identify individual cats in a multi-cat scenario, identify litter box maintenance events, and/or predict a number of insights unique to each cat/litter box event. Based on this information, a variety of events can be determined that describe the animal's behavior. For example, a determination can be made if the load sensor data is derived from cat behaviors and/or a person interacting with the litter box. If the behaviors are associated with a cat, a determination can be made if the cat is interacting with the inside or outside of the litter box. If the cat is inside the litter box, the identity of the cat and/or the cat's activity (urinating, defecating, etc.) can be determined. If the cat is outside the litter box, a variety of behaviors (e.g., rubbing the box, balancing on the edge of the box, etc.) can be determined. If the behaviors are associated with a person, it can be determined if the person is scooping the litter, adding litter, interacting with the smart litter box system, or the like. Furthermore, the smart litter box system can automatically track visit frequency, visit type (e.g., elimination vs. non-elimination), and/or animal weight across multiple visits. This historical information can be used to monitor animal weight, litter box visit frequencies, and/or elimination behaviors over time. This information, in some instances, can be combined with a variety of other data regarding the animal (e.g., age/life stage, sex, reproductive status, body condition, rate-of-change in weight or behavior, and the like), and furthermore, can be used to identify when changes occur and/or predict potential health or behavioral conditions affecting the animal.

In addition to identifying animal behaviors, the animal health monitoring system can advantageously provide early indicators of potential health conditions including, but not limited to, physical, behavioral and mental health of an animal. One example of physical health that can be monitored and diagnosed includes renal health. Renal health relates to various degrees of kidney disease, such as chronic kidney disease (CKD), acute kidney failure, etc. Chronic kidney disease, for example, be characterized by a sustained decrease in renal function over at least 3 months. It is not a single entity but a heterogeneous syndrome resulting in loss of functioning renal mass. In veterinary patients, congenital or acquired disorders can lead to development of CKD. Acute kidney damage (single or repeated episodes) secondary to urinary obstruction, nephrotoxins, pyelonephritis, or ischemic injury also can progress to CKD. It is a relatively common condition in cats, affecting roughly 2% of cats overall and chronic renal failure is diagnosed in approximately 8% of cats older than 10 years and in 15% of cats older than 15 years. The kidney plays a role in removing metabolic wastes from the bloodstream, regulating fluid and electrolyte balance, producing or activating hormones, controlling blood pressure, to name a few. Some symptoms of chronic kidney disease include frequent urination (as the cat is unable to hold water), high volume drinking of water, bacterial infections in the kidney and/or bladder, weight loss, decreased appetite, vomiting, diarrhea, bloody or cloudy urine, mouth ulcers, bad breath with an ammonia-like odor, a brownish colored tongue, a dry coat, constipation, etc. In further detail, cats presenting with CKD typically experience excessive urination (polyuria), frequent urination at night (nocturia) and shifts in defecation patterns to either constipation or diarrhea. For example, it is estimated that 40% of cats with CKD experienced excessive urination, less than 10% had inappropriate urination (periuria), and 3% had diarrhea. Cats with CKD have also been identified to be at an increased risk of severe constipation at times benefitting from emergency intervention. In accordance with examples of the present disclosure, many of these symptoms are discovered using a smart litter box system in accordance with the present disclosure.

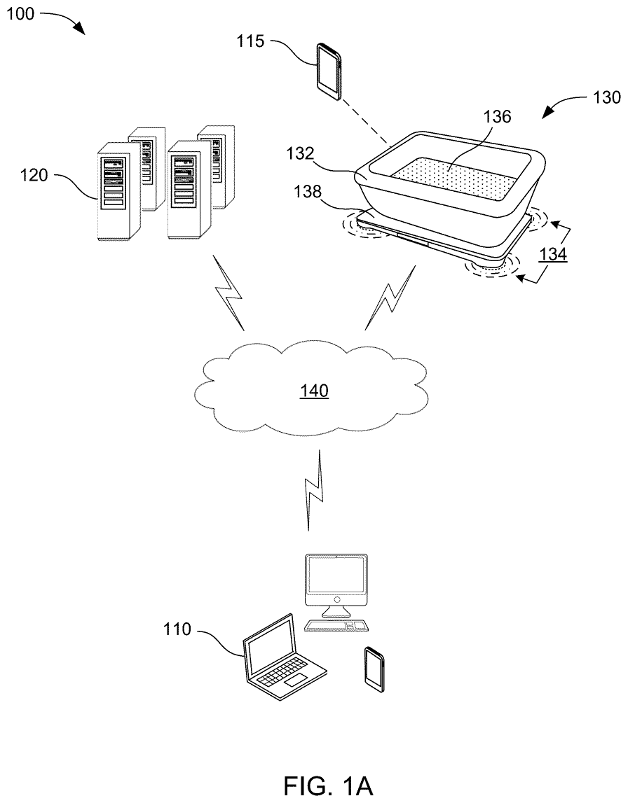

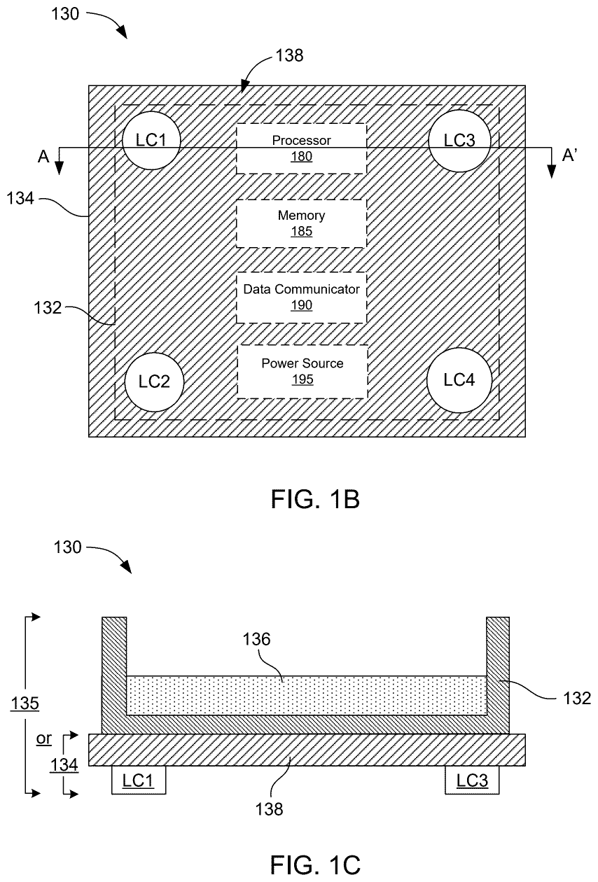

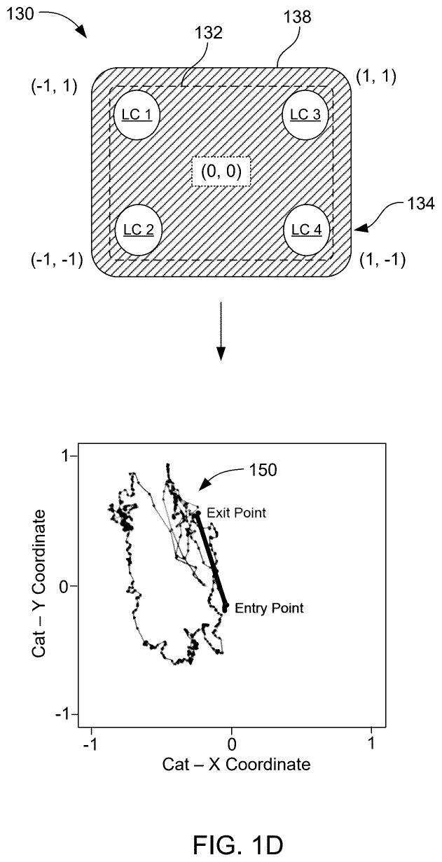

Turning now to the drawings included herewith, FIG. 1A schematically illustrates an animal health monitoring system 100. The animal health monitoring system can include client devices 110, an analysis server system 120, and/or a smart litter box system 130, which may be in communication with the client device(s) and/or the analysis server system via a network 140. In this example, the smart litter box system can include a litter box container 132 to receive litter 136 (shown in this example as containing the litter), and the litter box container can rest on top of a load monitoring device 134, which can include platform 138 equipped with a plurality of load cells (not shown, but shown as LC1-LC4 in FIGS. 1B-1D). However, there are other configurations that can be used. For example, the litter box container may be an off the shelf litter box that may be placed on the platform, the litter box container may be purpose built for the platform, or the litter box container may be integrated with or coupled to the platform. In the instance where the litter box container is integrated with the platform, then the “load monitoring device” itself can be configured to receive and contain litter. Notably, FIG. 1C illustrates a load monitoring device similar to that shown in FIG. 1A that is adapted to carry a separate litter box container, or alternatively, an integrated load monitoring container device 135 is shown that is integrated with the litter box container. In either instance, the smart litter box system would include the platform, the load cells, and a way to contain litter above the platform, whether that be integrated with the litter box container or not. In additional detail, the platform is depicted as generally rectangular in shape. However, the platform can be any shape such as a square, rectangle, circle, triangle, etc.

In further detail regarding the smart litter box systems 130 described herein, it is notable that the load monitoring device 134 (or the integrated load monitoring container device 135 shown in FIG. 1C) can obtain data regarding the interactions of animals and/or people with the smart litter box system due to a cat's interaction with the litter box container 132 and litter 136 contained therein. For example, interaction with the litter box container and litter can cause load sensors to sense pressure or load induced by the animal during this interaction. The load sensors (not shown, but shown as LC1-LC4 in FIG. 1B-1D) can be located in a position that does not disrupt the cat's natural behavior. The load sensors can be used to detect the presence of a cat in the litter box container and/or measure a characteristic of the cat when it is in the litter box container, e.g., movement, activity, behaviors, etc., and after it has left the litter box container, e.g., load change due to elimination and/or defecation, location of elimination and/or defecation, modification of litter distribution, etc. More specifically and by way of example, the load sensors can be positioned to track an animal's movements within or in contact with the litter and/or litter box container. The data captured using the load sensors can be used to determine animal elimination behaviors, behaviors other than elimination behaviors that may occur inside or outside of the litter box container (e.g., cats rubbing the litter box container), other environmental activities, etc.

Client devices 110 can include, for example, desktop computers, laptop computers, smartphones, tablets, and/or any other user interface suitable for communicating with the smart litter box systems. Client devices can obtain a variety of data from one or more smart litter box system 130, provide data and insights regarding one or more animals via one or more software applications, and/or provide data and/or insights to the analysis server systems 120 as described herein. The software applications can provide data regarding animal weight and behavior, track changes in the data over time, and/or provide predictive health information regarding the animals as described herein. In some embodiments, the software applications obtain data from the analysis server systems for processing and/or display.

Analysis server systems 120 can obtain data, return data, and/or be otherwise interactive with a variety of client devices 110 and/or load monitoring devices 134 (which may or may not be integrated with the litter box container 132) as described herein. In some aspects, the analysis server systems may be in the form of a single server or a plurality of interconnected servers. In other examples, the analysis server system may include a plurality of interconnected devices and/or may be in the form of a cloud-based server system. For example, multiple servers may be at a single location or may communicate with one another locally or may be interconnected over the network 140. The analysis server systems can provide data and insights regarding one or more animals and or transmit data and/or insights to the client devices as described herein. These insights can include, but are not limited to, insights regarding animal weight and behavior, changes in the data over time, and/or predictive health information regarding the animals as described herein. In a number of embodiments, the analysis server systems obtain data from multiple client devices and/or load monitoring devices of the smart litter box systems, identify cohorts of animals within the obtained data based on one or more characteristics of the animals, and/or determine insights for the cohorts of animals. The insights for a cohort of animals can be used to provide recommendations for a particular animal that has characteristics in common with the characteristics of the cohort. In some examples, the analysis server systems can provide a portal (e.g., a web site) for pet owners and/or veterinarians to access information regarding the health of a particular animal.

In operation, the smart litter box systems 130 (or the load monitoring device 134 which includes the load sensors) can transmit data to the client devices 110 and/or analysis server systems 120 for processing and/or analysis. In some examples, the smart litter box systems can communicate directly with a non-network client device 115 without sending data through the network 140. The term “non-network” client device does not infer it is not also connected via the cloud or other network, but merely that there is a wireless or wired connection that can be present directly with the smart litter box system. For example, the smart litter box systems and the non-network client device can communicate via Bluetooth. In some embodiments, the smart litter box systems process the load sensor data directly. In many embodiments, the smart litter box systems utilize the load sensor data to determine if the smart litter box system is unbalanced. In this instance, automatic or manual adjustment of one or more adjustable feet can rebalance the smart litter box system. In this way, the smart litter box systems can adjust their positioning to provide a solid platform for the waste elimination area.

Any of the computing devices shown in FIG. 1A (e.g., client devices 110, analysis server systems 120, smart litter box systems 130, etc.) can include a single computing device, multiple computing devices, a cluster of computing devices, and the like. A computing device can include one or more physical processors communicatively coupled to memory devices, input/output devices, and the like. As used herein, a processor may also be referred to as a central processing unit (CPU). The client devices can be accessed by the animal owner, a veterinarian, or any other user. Additionally, as used herein, a processor can include one or more devices capable of executing instructions and encoding arithmetic, logical, and/or I/O operations. In one illustrative example, a processor may implement a Von Neumann architectural model and may include an arithmetic logic unit (ALU), a control unit, and a plurality of registers. In many aspects, a processor may be a single core processor that is typically capable of executing one instruction at a time (or process a single pipeline of instructions) and/or a multi-core processor that may simultaneously execute multiple instructions. In some examples, a processor may be implemented as a single integrated circuit, two or more integrated circuits, and/or may be a component of a multi-chip module in which individual microprocessor dies are included in a single integrated circuit package and hence share a single socket. As discussed herein, a memory refers to a volatile or non-volatile memory device, such as RAM, ROM, EEPROM, or any other device capable of storing data. Input/output devices can include a network device (e.g., a network adapter or any other component that connects a computer to a network), a peripheral component interconnect (PCI) device, storage devices, disk drives, sound or video adaptors, photo/video cameras, printer devices, keyboards, displays, etc. In several aspects, a computing device provides an interface, such as an API or web service, which provides some or all of the data to other computing devices for further processing. Access to the interface can be open and/or secured using any of a variety of techniques, such as by using client authorization keys, as appropriate to the requirements of specific applications of the disclosure.

In further detail, the network 140 can include a LAN (local area network), a WAN (wide area network), telephone network (e.g., Public Switched Telephone Network (PSTN)), Session Initiation Protocol (SIP) network, wireless network, point-to-point network, star network, token ring network, hub network, wireless networks (including protocols such as EDGE, 3G, 4G LTE, Wi-Fi, 5G, WiMAX, and the like), the Internet, and the like. A variety of authorization and authentication techniques, such as username/password, Open Authorization (OAuth), Kerberos, SecureID, digital certificates, and more, may be used to secure the communications. It will be appreciated that the network connections shown in the example computing system 100 are illustrative, and any means of establishing one or more communication links between the computing devices may be used.

FIG. 1B is a bottom plan view and FIG. 1C is a side cross-sectional view of a smart litter box system 130 taken along section A-A′, which can be used in the animal health monitoring systems and methods of the present disclosure. This example is shown to illustrate components that may be part of the smart litter box system, and more specifically, the load monitoring device 134 or the integrated load monitoring container device 135.

In this example, the smart litter box system 130 is depicted as having four load sensors LC1, LC2, LC3, and LC4. It should be appreciated that the smart litter box system can be capable of functioning with three or more load sensors and is not limited to four load sensors. Individual load sensors of the four load sensors are associated with the platform 138 and separated from one another and receive pressure input independent of one another. In some examples, the platform can be a triangular shape and can be associated with three load sensors. The triangular shape allows the smart litter box system to be easily placed in a corner of a room.

The smart litter box system 130 can include a processor 180 and a memory 185. The processor and memory can be capable of controlling the load sensors and receiving load data from the load sensors. The load data can be stored temporarily in the memory or long term. The data communicator 190 can be capable of communicating the load data to another device. For example, the data communicator can be a wireless networking device with employee wireless protocols such as Bluetooth or Wi-Fi. The data communicator can send the load data to a physically remote device capable of processing the load data such as the analysis server systems 120 of FIG. 1A. The data communicator can also transmit the data over a wired connection and can employ a data port such as a universal serial bus port. Alternatively, a memory slot can be capable of housing a removable memory card where the removable memory card can have the load data stored on it and then physically removed and transferred to another device for upload or analysis. In one embodiment, the processor 180 and memory 185 are capable of analyzing the load data without sending the load data to a physically remote device such as the analysis server systems. The smart litter box system 130 can also include a power source (not shown), such as an AC power source, e.g., wired power source that plugs into an electrical wall outlet, or a DC power source, e.g., replaceable battery or rechargeable battery. The power source can be a combination of a battery and a wired power source. The smart litter box system may be built without a camera or image capturing device and can be used without other equipment, such as an RFID collar or other collar that senses movement or activity.

In operation, a cat may enter its litter box container 132, find a spot within the litter 136 to eliminate, cover the elimination, and exit the litter box. An animal health monitoring system can track the activity of the cat while in the smart litter box system using multiple load sensors LC1-LC4 that measure the distribution of the cat's weight and the overall weight of the system. This data can be processed to identify specific cat characteristics, derive features related to the cat behaviors, e.g., location of elimination, duration, movement patterns, force of entry, force of exit, volatility of event, and the like. A variety of events can be determined based on these characteristics and features. In some examples, one or more machine learning algorithm or classifier can be used to determine these events. However, in accordance with the present disclosure, machine learning can be used in connection with these smart litter box systems to predict whether or not a cat may be suffering from one or more renal disease or condition, e.g., acute kidney failure, even in the early stages before symptoms become more obvious to the pet owner or veterinarian. Furthermore, the smart litter box system can also account for interactions such as false triggers, human interactions, cat out of box interactions, etc., which may be discarded by the machine learning algorithms as not providing relevant information related to whether or not the cat is suffering from a renal disease or condition. That data is typically collected while the cat is interacting with the litter inside the litter box container.

To illustrate the sensitivity and possibilities of using multiple load sensors which independently record their own weights or loads over time, FIG. 1D illustrates an example of location tracking of an animal's movement path 150 over time. In this example, the animal's movement path within the litter box can be described from the entry to exit of a litter box. The movement path can be tracked using the animal's center of gravity. In this example, an animal health monitoring system may be used that includes a smart litter box system 130 including a litter box container 132, a platform 138, and multiple load sensors, e.g., LC1, LC2, LC3, and/or LC4, each located proximate to a corner of the platform in this instance. The smart litter box system would carry a litter box with contained litter thereon (not shown). For convenience, a coordinate system can be defined where the center of the platform (which may be aligned with a center of the litter box) is defined as (0, 0), a first corner approximately where LC1 resides is defined as (−1, 1), a second corner approximately where LC2 resides is defined as (−1, −1), a third corner approximately where LC3 resides is defined as (1, 1), and a fourth corner approximately where LC4 resides is defined as (1, −1).

In this example, the initial center of gravity of the animal health monitoring system can be calculated based on the tare (empty) weight of the animal health monitoring device with the contained litter carried thereon. When the animal enters the litter box, each load sensor can obtain a different load measurement depending on the animal's location within the litter box. At a given time, the center of gravity of the animal can be calculated based on the measurement from each of the load sensors. Movement path 150 shows various locations of the center of gravity of the animal while in the litter box resting on top of the smart litter box system, including approximate entry and exit points. As individual animals have their own unique personality, habits and routines, the general movement of the animal during a particular class of events is typically unique to that animal. In this way, the animal's movement data can be used as a signature to identify the animal during a particular event. Furthermore, with individual cats, by tracking the animal's center of gravity, the location of the animal within the litter box can be determined for each phase and/or each feature within the event.

In addition to an animal's movement patterns for a particular event, a variety of other characteristics of the event can be used to classify events and/or identify particular animals. These characteristics include, but are not limited to, the weight of the animal, the time at which the animal typically performs a particular class of event, the location of the animal during one or more phases of the event, covering behavior (e.g., covering in place, exiting and returning to the litter box to cover, standing halfway in the litter box to cover, paw the litter box, and the like), climbing over the edge of the litter box versus jumping into the litter box, total duration of inside box activity, litter box preference for one unit over another in multi-unit environments, typical weight of elimination, times of entry/exit before eliminating, time spent digging before/after eliminating, force used to cover elimination, speed of paw movements for covering, patterns of movement within the litter box (e.g., clockwise and/or counterclockwise movement), consistency in choice of elimination spot, and ordering of cats entering the box in a multiple cat home. In some examples, as many pet owners have multiple animals that utilize the same litter box, the animal health monitoring systems 100 of the present disclosure can be tuned or adapted to distinguish between multiple animals using the same litter box. This and other information can be used by trained and tested/validated artificial intelligence or machine learning algorithms to detect behaviors that may be indicative of renal health of the animal, as described in greater detail hereinafter.

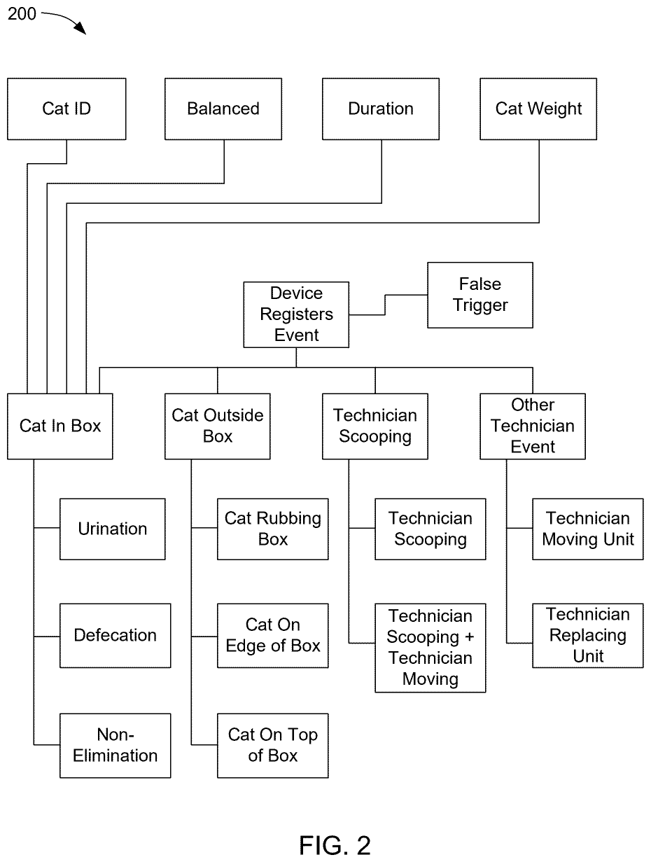

FIG. 2 illustrates one possible conceptual overview of example events 200 occurring within a smart litter box system according to examples of the present disclosure. For example, some events can include false triggers, cat in box events, cat outside box events, scooping events, etc. A false trigger can indicate that some data was obtained from the load sensors, but no corresponding event was occurring. Cat in box events can include elimination events (e.g., urination and/or defecation) and non-elimination events. When a cat in box event is detected, a variety of characteristics of the cat can be determined. These characteristics can include, but are not limited to, cat identification (cat ID), balance of the device, duration of the event, weight of the cat, intensity of the event, etc. Cat outside box events can include the cat rubbing the litter box, the cat standing on the edge of the litter box, and/or the cat standing on top of the litter box. In connection with the present disclosure, as cat in box events are typically more useful in predicting renal health of a cat, activities that relate to renal conditions can be used as features for predicting renal health of the cat. Examples of such activity may include the recording and/or characterization of urination events, weight variability, duration of interacting with the smart litter box system, duration of finding/digging in the litter, intensity of litter covering, etc.

It is noted that false triggers can be removed from the machine learning algorithms automatically by properly training the artificial intelligence to detect these type of activities and exclude them from the relevant interactions between the cat and the smart litter box system. For example, scooping events can include events where litter and/or waste are being removed from the litter box by a technician. Scooping events can include scooping the litter box, adding litter to the litter box, and moving the litter box. For example, a user may pull the litter box towards them and/or rotate the litter box to gain more ready access to all portions of the litter box for complete waste removal. Other events can include moving of the animal health monitoring system and/or litter box by a user. For example, a user can move the animal health monitoring system from one location to another, replace the litter box located on top of a smart litter box system, remove or replace a lid on the litter box, or the like. Other non-limiting possible interactions are shown by way of example in FIG. 2. Notably, the order and/or arrangement of some of the blocks may be changed, certain blocks may be combined with other blocks, one or more blocks may be repeated, and/or some of the blocks described may be excluded. FIG. 2 merely provides an example of how the animal health monitoring system may be programmed and/or operated, e.g., by processing logic that may include hardware (circuitry, dedicated logic, etc.), software, or a combination of both. These systems and/or methods described herein may thus be implemented as a method and executed as instructions on a machine, where the instructions are included on at least one computer readable medium or one non-transitory machine-readable storage medium, for example.

In further detail, one or more machine learning classifiers or machine learning algorithms can be used to analyze the load data to identify and/or label events within the load data. Based on the labels, the events and/or animals can be classified. It should be readily apparent to one having ordinary skill in the art that a variety of machine learning classifiers can be utilized including (but not limited to) decision trees (e.g. random forests, catboost), k-nearest neighbors, support vector machines (SVM), neural networks (NN), recurrent neural networks (RNN), convolutional neural networks (CNN), and/or probabilistic neural networks (PNN). RNNs can further include (but are not limited to) fully recurrent networks, Hopfield networks, Boltzmann machines, self-organizing maps, learning vector quantization, simple recurrent networks, echo state networks, long short-term memory networks, bi-directional RNNs, hierarchical RNNs, stochastic neural networks, and/or genetic scale RNNs. In a number of embodiments, a combination of machine learning classifiers can be utilized. More specific machine learning classifiers when available, and general machine learning classifiers at other times, can further increase the accuracy of predictions.

Individual features may be used, such as by the use of a machine learning classifier or machine learning algorithm for classification, and in some examples, multiple features may be used simultaneously by the machine learning classifier or multiple different machine learning classifiers. Classifying events can include identifying the features and a confidence metric indicating the likelihood that the labels correspond to the ground truth of the events (e.g., the likelihood that the labels are correct). These predictions can be determined based on the features, phase, and/or a variety of other data, and the features that are developed during training, for example, can be used to classify behaviors using one or more machine learning classifiers or machine learning models. For example, a variety of features can be developed or created in the time domain and/or the frequency domain. These features include, but are not limited to, the standard deviation of the load, a length of a flat spot, a crossover count of mean, a unique peak count, a distinct load value count, a ratio of distinct load values to event duration, a count of max load changes in individual sensors, a medium load bin percentage, a high load bin percentage, high load bin volatility, high load bin variance, automatic correlation function lag or latency, curvature, linearity, count of peaks, energy, minimum power, a power standard deviation, maximum power, largest variance shift, a maximum Kulback-Leibler divergence, a Kulback-Leibler divergence time, spectral density entropy, automatic correlation function differentials, and/or a variation of an autoregressive model. Behaviors can thus be classified based on a correlation with the classified features. For example, the selected features can be used as inputs to machine learning classifiers to classify the behaviors. The classified behaviors can include a prediction indicating the type of behavior and/or a confidence metric indicating the likelihood that the prediction is correct. The machine learning classifiers can be trained on a variety of training data indicating animal behaviors and ground truth labels with the features as inputs. As the present disclosure is related primarily to the prediction of renal health of cats, features can be selected that provide improvement in separating out predictions of renal cats versus healthy cats. Furthermore, events identified using the load data may be categorized based on the identified (or created) features and/or the phase data. In some examples, the events can be categorized based on the confidence metric indicating the likelihood that one or more events have been correctly classified. For example, the events can be classified into elimination events, scooping events, cat sitting on litter box events, and/or any of a variety of other events as described herein. In further detail, an event can cause changes in the overall state of the animal health monitoring system. For example, adding litter, changing litter, and scooping events can cause the overall weight of the litter box to change. In these cases, the animal health monitoring system can recalibrate its tare weight to maintain the accurate performance of the animal health monitoring system. Once a prediction is made, in some examples, a notification can be transmitted to a client device from the analysis server over the network. The client device may be in possession of a pet owner, pet caregiver, veterinarian, or the like, for example.

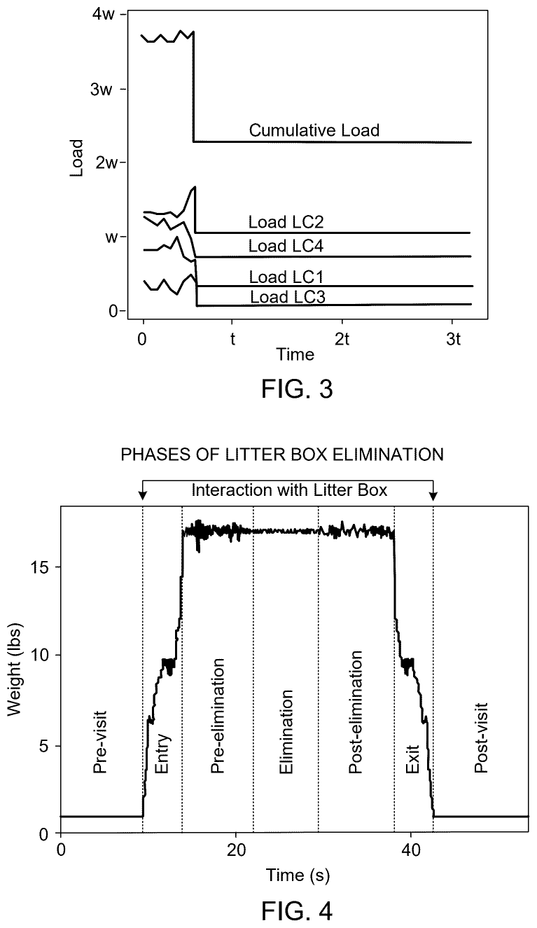

FIG. 3. illustrates an example graph depicting each load cell (of 4 load cells in this example) of the smart litter box system independently sensing loads over time, and furthermore, that a cumulative load value can be generated using the load data from the independent load cells. Thus, each of the four (4) load cells in this example independently collects its own load data, e.g., pounds, kilograms, etc., over a period of time, e.g., seconds. With each load cell independently collecting its own load data, various cat interactions can be understood and characterized for purposes of renal disease or condition predictions. Thus, load data can be analyzed as cumulative load (or total load), individual load per load sensor, and/or at a phase level via a phase separation algorithm(s) separating the load data into phases. Example phases may include pre-elimination (e.g. entering, finding, digging), elimination (e.g. urination, defecation), and post-elimination (e.g. covering, exiting). In addition to these phases (and events that may occur within certain phases), the animal's behavior and location (See FIG. 1D by way of example) can also be determined.

FIG. 4 illustrates a cat interaction with a litter box during an elimination event. In this example, the cumulative load values are shown, but it is understood that this data can be based on the collection of load data from multiple independent load sensors, as shown by way of illustration in FIG. 3. For example, the activity associated with a cat's interaction with the smart litter box system can be represented as a graph that has a variety of peaks, valleys, flat spots, etc., which can be interpreted to identify phases, events, and ultimately to select features based on renal health of a cat. Load values over time collected using the animal health monitoring systems described herein, including the use of the smart litter box system associated with multiple independent load cells, can be used to record behaviors of the cat interacting with the litter in the smart litter box system. Those behaviors, including the times of events (time domain), frequency of events (frequency domain), intensity of events, etc., can be used to characterize what, how long, how intense, etc., the cat is conducting various events within the smart litter box system. To illustrate, load data can include information in the time domain, in frequency domain, or both. In some examples, the load data can be transformed from time domain data to frequency domain data. For example, time domain data can be transformed into frequency domain data using a variety of techniques, such as a Fourier transform. Similarly, frequency domain data can be transformed into time domain data using a variety of techniques, such as an inverse Fourier transform. In some embodiments, time domain features and/or frequency domain features can be identified based on particular peaks, valleys, and/or flat spots within the time domain data and/or frequency domain data as described herein. Furthermore, features related to intensity can be determined based on the load data collected as well, which includes both time domain and frequency domain properties.

Regarding a typical cat elimination event as shown in FIG. 4, there may be an initial increase in weight as the cat enters the litter box (entry), a period of motion where the cat moves within the litter box (pre-elimination, e.g., entering, digging, finding, etc.), a pause in activity while the cat performs the elimination event (elimination, e.g., urination and/or defecation), a second period of motion as the cat buries the elimination (post-elimination, e.g., covering, exiting, etc.), and a decrease in weight of the litter box as the cat exits the litter box (exit). Also shown in FIG. 4 is the period of time prior to (pre-visit) and after (post-visit) the cat enters and exits the litter box container, respectively. As described in more detail herein, flat spots in the activity typically correspond to actual voiding of the bladder or bowel during elimination events. In some examples, the duration of particular events provides an indication of the activities occurring during the event. For example, most mammals take approximately 20 seconds to empty their bladder and non-elimination events are typically shorter than urination events, which are shorter than defecation events. Additionally, changes in weight of the litter box after an event occurs can be an indicator of the event that occurred as urination events typically result in a larger weight increase than defecation events. Furthermore, other indicators related to healthy elimination may relate to covering of the elimination (amount of time, intensity or energy used, etc.), the amount of time and/or movement to find an elimination spot, the amount of time preparing the elimination spot, e.g. digging in the litter or other energy spent prior to elimination, the amount of time and energy covering the elimination, movement during elimination, e.g., scooting, hip thrusts, etc., step/slope detection on a single load sensor during a flat spot, and others. This type of activity information can be informative in developing features for predicting the renal health of cats in accordance with examples of the present disclosure.

In further detail, each individual visit by a cat to the smart litter box system may include multiple events that can occur during one or more phases (or phases of litter box elimination), such as the five (5) phases shown by way of example in FIG. 4, e.g., entry, pre-elimination, elimination, post-elimination, and exit, using the collected load data. With that stated, some systems may use fewer or more than these five (5) phases. For example, some systems may use three (3) phases, e.g., pre-elimination, elimination, and post-elimination. Regardless, the load data can be evaluated to determine the “flattest” spot along the load data collected, which typically corresponds to an elimination event (e.g., occurring in the elimination phase), with data occurring prior to the flat spot being the pre-elimination phase and data occurring after the flat spot being the post-elimination phase. With that stated, in some examples, consecutive sliding windows can be used to analyze the load data, and sliding windows with minimal difference (e.g., a difference below a threshold value pre-determined and/or determined dynamically) in variance may be grouped together as potential flat spots, indicating the elimination phase or an elimination event. With this process, the group of consecutive windows with the largest number of samples may typically indicate the flat spot for the event where elimination may be occurring. This same process can be used to evaluate and identify other phases and/or events, and features can be selected that may provide predictions acceptable renal precision and/or healthy recall.

Load values collected over time (sensed by the load cells of the smart litter box system and analyzed by the analysis server), for example, can be used to detect behaviors indicative of various phases and/or events occurring during a single visit to the smart litter box system. Furthermore, phase and event data collected can be used to establish features that may be identifiable from the data suitable for making predictions. Correctly selected features that may be more indicative of poor renal health (based on analysis of the load values) can be very predictive of whether a cat is renal or healthy. Thus, features can be selected to assist with making accurate predictions of renal health based on events that may occur at any of the phases of litter box elimination. Furthermore, events can be determined by analyzing the total load data (cumulative from all of the load sensors) and/or the load data for each of the individual load sensors. Specific events can be identified by using potential features in the load data for each of the load sensors and aggregating the potential features (if more than one is used) to identify the most effective features or combinations of features within the total load data. This aggregation can be any mathematical operation including, but not limited to, sums and averages of the potential features.

Features can be developed for processing the load data for each phase or for multiple phases to identify particular behaviors that occur during that phase. The load data can be analyzed in both the time domain and the frequency domain. Time domain features include, but are not limited to, mean, median, standard deviation, range, autocorrelation, and the like. The time domain features are created as inputs for the machine learning classifier. Frequency domain features include, but are not limited to, median, energy, power spectral density, and the like. The frequency domain features are created as inputs for the machine learning classifier. Features that are particularly related to predicting renal disease can be selected based on their ability to provide better renal precision and/or healthy.

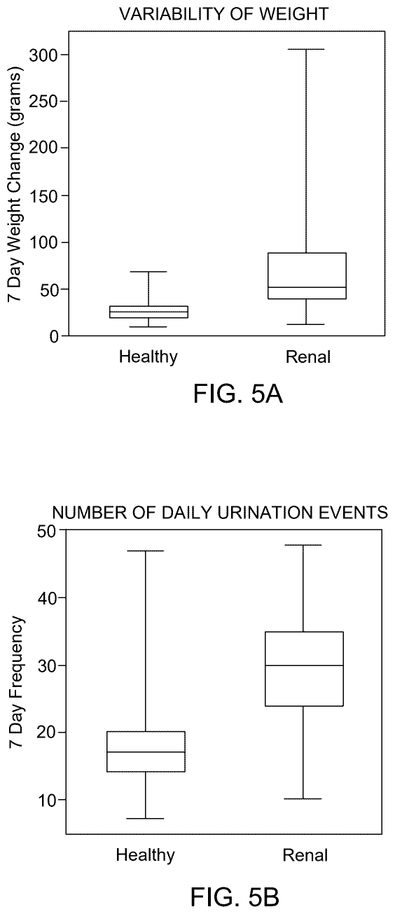

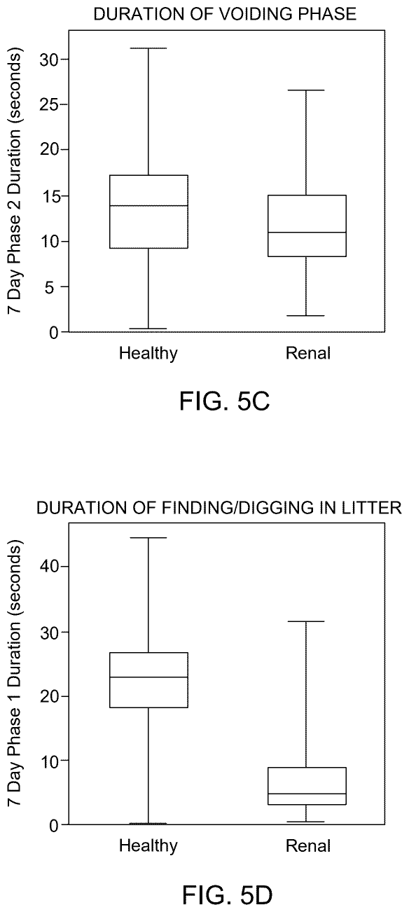

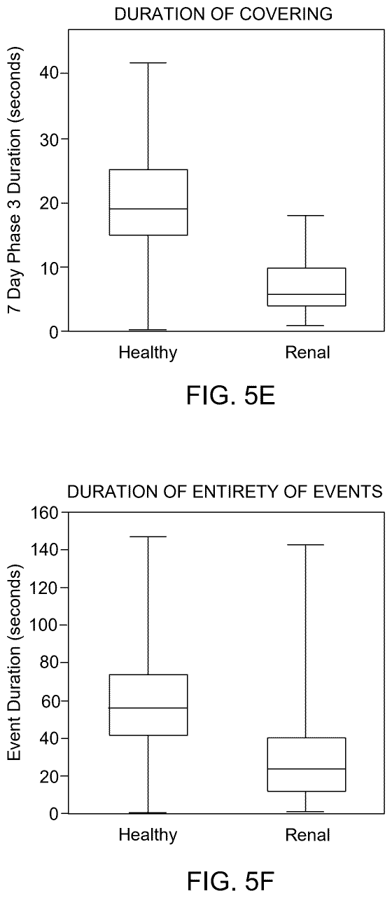

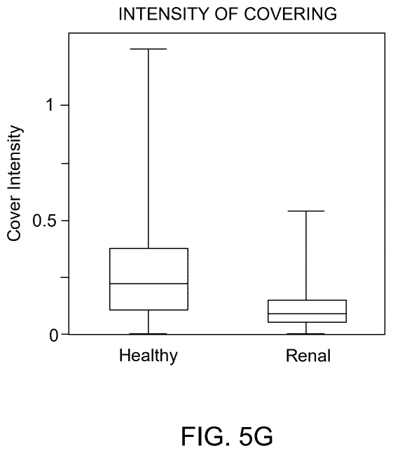

After review of many features that can be picked up by the animal health monitoring systems of the present disclosure, and along with the training of different machine learning models with various features and feature combinations, it was found that there are a handful of features that can be detected from load data that may be particularly helpful in predicting if a cat is renal (via renal precision) or healthy (via healthy recall). Using renal precision as an example, there were several example features identified that may be used to predict that a cat has compromised renal health. These renal precision features are shown in Table 1, with corresponding data collected for several of these features shown by way of example in FIGS. 5A-5G, as follows:

TABLE 1
Features for Predicting Renal Precision of Cats

| Feature ID | Feature Description for Renal Precision | Data |
|---|---|---|
| (i) | increased variability of weight | FIG. 5A |
| (ii) | increased number of daily urination events | FIG. 5B |
| (iii) | similar duration of voiding phase of elimination events | FIG. 5C |
| (iv) | decreased duration of finding/digging in litter | FIG. 5D |
| (v) | decreased duration of covering | FIG. 5E |
| (vi) | decreased duration of entirety of events | FIG. 5F |
| (vii) | decreased intensity of covering | FIG. 5G |
| (viii) | decreased intensity of finding/digging | Not Shown |
| (ix) | decreased number of defecation events | Not Shown |
| (x) | increased number of litter box visits | Not Shown |
| (xi) | decreased weight of elimination | Not Shown |

Values are based on 7 day averages.

Data collected from the multiple load cells of the smart litter box system can be based on multiple day data and then averaged for a specific cat in the study. Any number of days suitable for collecting reliable data can be used, e.g., from a few days to several months, but the data collected in FIGS. 5A-5G was based on a 7 day study of these six features and others (which were not as predictive of renal health). In additional detail, in addition to the features that include data as illustrated herein in FIGS. 5A-5G, there are also other features that can also assist in predicting renal health of cats which are not shown, e.g., features (viii)-(x). Any of these features (or others) can be used alone or in combination with other features to reasonably accurately predict the renal health of cats. In accordance with this, a reasonably accurate prediction that a cat is suffering from compromised renal health can be at least about 75% in accuracy as it relates to renal precision, for example. In some examples, it has been found that models that utilize feature (ii) (increased number of daily urination events) alone or in combination with other features using at least a 0.5 probability threshold typically results in a renal precision prediction of at least about 75%. By adding other features to the model, by increasing the probability threshold, e.g., to 0.7, and/or by adding an overrule, e.g., a renal precision heuristic overrule, renal precision predictions can be further increased. As a note, the data shown in FIG. 5C illustrates an example of a feature that was evaluated and found to be less helpful predicting the renal health of cats, particularly when used alone, due to the significant overlap in time of voiding. Thus, this feature may be usable for marginal improvement in data.

To train and test artificial intelligence to detect cat behavioral indicators that may be associated with renal health, many features (or variables) that can be detected by the smart litter box systems of the present disclosure can be considered. Examples of data that can be collected include precision, e.g., renal precision or healthy precision; recall, e.g., renal recall or healthy recall; F1 score, e.g., renal cat F1 score or healthy cat F1 score; weighted F1; etc. After some evaluation, it was determined that focus on renal precision and healthy recall provided predictive data that was very helpful in making cat health predictions. With that stated, in some instances, healthy precision and/or renal recall can be used alternatively or in addition to one or more of renal precision and/or healthy recall, particularly if the probability of correct predictions can be increased. In further detail, it was found that various features can be selected that improve renal precision and healthy recall resulting from cat interaction with a smart litter box system as described herein.

“Renal precision” in particular relates to the true number of renal predictions relative to the total number of renal predictions. As an example, a renal precision value of at least about 75% can be considered to be acceptable for purposes of reasonably and reliably predicting renal health issues, even with early onset renal diseases that are difficult to predict, e.g., at least about 75% renal precision. “Healthy recall,” on the other hand, relates to the true number of healthy predictions relative to the total number of healthy cats. For simplification, Formulas 1 and 2 below provide general formulas associated with renal precision and healthy recall, as follows:

$$Renal Precision = True Number of Renal Predictions / Total Number of Renal Predictions \qquad Formula 1$$

$$Healthy Recall = True Number of Healthy Predictions / Total Number of Healthy Cats \qquad Formula 2$$

In making predictions for the renal health of cats, in addition to the variability of features selected, other tools can be used to increase renal precision and/or healthy recall. In particular, increasing the probability threshold used to obtain a reliable prediction can also lift renal precision, e.g., increase the accuracy of a renal prediction. However, increasing the probability threshold too much can be counter-productive, as it could lead to difficulty in obtaining any prediction that a cat may be experiencing renal disease. Likewise, setting a probability threshold that is too low may pick up too many cats that are not experiencing renal disease. Furthermore, when a goal may include predicting renal disease at an early stage before symptoms become prominent, e.g., which may be too late for effective treatment, a balance between a reasonable probability threshold that may pick up some false positives and a more stringent probability threshold that may not pick up the earlier stages of renal disease can be struck. In accordance with examples of the present disclosure, a probability threshold from about 0.5 to about 0.9 can be effective for reasonably accurately predicting whether a cat is renal with acceptable renal precision and/or whether a cat is healthy with acceptable healthy recall.

Table 2A below provides data collected for various individual and combinations of features at various probability thresholds, e.g., 0.5 and 0.7, as follows:

TABLE 2A
Renal Precision and Healthy Recall Predictions with
0.5 or 0.7 Probability Threshold (7 Day Averages)

| | Renal Precision | | Healthy Recall | |
|---|---|---|---|---|
| *Feature(s) ID | 0.5 | 0.7 | 0.5 | 0.7 |
| (i) | 52.3% | 48.7% | 82.2% | 96.4% |
| (ii) | 78.7% | 83.1% | 92.1% | 95.6% |
| (iii) | 34.9% | 17.1% | 83.5% | 97.9% |
| (iv) | 39.7% | 62.8% | 77.6% | 95.6% |
| (v) | 59.5% | 57.8% | 88.4% | 93.9% |
| (vi) | 65.8% | 72.6% | 95.0% | 97.0% |
| (vii) | 40.8% | 40.0% | 80.6% | 98.5% |
| (viii) | 45.6% | 77.8% | 87.7% | 97.9% |
| (i), (ii) | 82.5% | 88.3% | 93.6% | 96.3% |
| (ii), (iii) | 73.6% | 78.6% | 89.6% | 93.3% |
| (ii), (iv) | 84.6% | 90.5% | 93.9% | 96.7% |
| (ii), (v) | 71.5% | 80.0% | 89.6% | 93.9% |
| (ii), (vi) | 72.3% | 74.7% | 90.5% | 92.9% |
| (ii), (vii) | 76.2% | 83.4% | 90.5% | 94.6% |
| (ii), (viii) | 75.9% | 83.4% | 90.9% | 94.7% |
| (i), (ii), (iv) | 83.1% | 88.1% | 93.6% | 96.0% |
| (ii)-(iv) | 75.2% | 80.6% | 89.7% | 93.2% |
| (ii), (iv), (v) | 76.9% | 81.4% | 90.7% | 93.3% |
| (ii), (iv), (vi) | 74.6% | 79.7% | 90.6% | 93.4% |
| (ii), (iv), (vii) | 81.6% | 90.1% | 92.0% | 96.3% |
| (ii), (iv), (viii) | 83.4% | 88.7% | 94.0% | 96.6% |
| (i), (ii), (iv), (vi) | 82.1% | 86.3% | 92.5% | 94.9% |
| (ii)-(iv), (vii) | 81.3% | 85.7% | 92.1% | 94.9% |
| (ii), (iv), (v), (vii) | 81.6% | 86.3% | 92.6% | 95.1% |
| (ii), (iv), (vi), (vii) | 77.8% | 80.5% | 91.0% | 92.9% |
| (ii), (iv), (vii), (viii) | 86.2% | 91.7% | 94.8% | 97.2% |
| (i), (ii), (iv), (vii), (viii) | 87.2% | 90.6% | 95.2% | 96.9% |
| (ii)-(iv), (vii), (viii) | 84.8% | 89.5% | 94.4% | 96.6% |
| (ii), (iv), (v), (vii), (viii) | 88.4% | 92.8% | 95.9% | 97.7% |
| (ii), (iv), (vi)-(viii) | 82.2% | 86.6% | 93.4% | 95.6% |
| (i)-(iv), (vii), (viii) | 84.3% | 88.8% | 94.2% | 96.4% |
| (i), (ii), (vi), (v), (vii), (viii) | 87.0% | 92.9% | 95.1% | 97.6% |
| (i), (ii), (vi), (vi)-(viii) | 83.8% | 87.4% | 93.8% | 95.8% |
| (i)-(v), (vii), (viii) | 84.4% | 89.5% | 94.4% | 96.7% |
| (i), (ii), (iv)-(viii) | 83.7% | 87.8% | 93.8% | 95.9% |
| (i)-(viii) | 81.8% | 86.9% | 93.2% | 95.8% |

*Features characterized as “increased” or “decreased” for Renal Precision data, as follows: (i) increased variability of weight; (ii) increased average number of daily urination events; (iii) similar duration of voiding phase of elimination events; (iv) decreased duration of finding/digging in litter; (v) decreased duration of covering; (vi) decreased duration of entirety of events; and/or (vii) decreased intensity of covering; (viii) decreased intensity of finding/digging.

In further detail, in addition to adjusting the probability threshold to improve/increase renal precision of the machine learning models described herein, by adding an overrule to the model, e.g., a renal precision heuristic overrule, the renal precision can sometimes be further improved. Thus, an “overrule” as used herein refers to a supplementary check for a prediction(s) that overrides the prediction that a cat is renal if certain criteria is not met. An example of an overrule that can be used includes a renal prediction heuristic overrule that adds an additional filter or check to the data before making the final prediction. For example, in the data collected and shown hereinafter, an overrule check showing compromised renal health would not be predicted unless the cat also showed an increased in the number of daily urinations, as this particular behavior was not easily explainable by other types of behavioral changes.

TABLE 2B
Renal Precision and Healthy Recall Predictions with
0.5 or 0.7 Probability Threshold (7 Day Averages
with Renal Prediction Heuristic Overrule Applied)

| | Renal Precision | | Healthy Recall | |
|---|---|---|---|---|
| Feature(s) | 0.5 | 0.7 | 0.5 | 0.7 |
| (i) | 85.0% | 83.0% | 97.1% | 99.5% |
| (ii) | 79.4% | 83.3% | 92.4% | 95.6% |
| iii | 63.4% | 63.6% | 95.6% | 99.7% |
| (iv) | 77.5% | 95.8% | 96.8% | 99.7% |
| (v) | 86.0% | 89.6% | 97.6% | 99.2% |
| (vi) | 82.9% | 86.1% | 98.2% | 98.8% |
| (vii) | 69.6% | 66.7% | 95.7% | 99.6% |
| (viii) | 80.2% | 98.2% | 97.6% | 99.9% |
| (i), (ii) | 83.7% | 88.6% | 94.2% | 96.4% |
| (ii), (iii) | 77.6% | 79.9% | 91.6% | 93.8% |
| (ii), (iv) | 89.2% | 92.8% | 96.1% | 97.7% |
| (ii), (v) | 80.2% | 85.6% | 93.6% | 95.9% |
| (ii), (vi) | 75.8% | 77.7% | 92.1% | 94.0% |
| (ii), (vii) | 79.3% | 83.4% | 92.3% | 94.7% |
| (ii), (viii) | 80.3% | 85.5% | 93.0% | 95.6% |
| (i), (ii), (iv) | 89.6% | 92.3% | 96.5% | 97.6% |
| (ii)-(iv) | 82.4% | 84.4% | 93.6% | 94.9% |
| (ii), (iv), (v) | 88.8% | 89.7% | 96.2% | 96.7% |
| (ii), (iv), (vi) | 84.4% | 85.4% | 95.1% | 95.7% |
| (ii), (iv), (vii) | 91.6% | 93.9% | 96.9% | 97.9% |
| (ii), (iv), (viii) | 90.5% | 92.5% | 96.9% | 97.9% |
| (i), (ii), (iv), (vi) | 92.2% | 93.8% | 97.2% | 97.9% |
| (ii)-(iv), (vii) | 90.1% | 91.0% | 96.6% | 97.2% |
| (ii), (iv), (v), (vii) | 92.2% | 94.3% | 97.3% | 98.2% |
| (ii), (iv), (vi), (vii) | 86.8% | 87.8% | 95.5% | 96.1% |
| (ii), (iv), (vii), (viii) | 94.3% | 96.3% | 98.1% | 98.8% |
| (i), (ii), (iv), (vii), (viii) | 94.2% | 96.5% | 98.1% | 98.9% |
| (ii)-(iv), (vii), (viii) | 92.8% | 95.1% | 97.6% | 98.6% |
| (ii), (iv), (v), (vii), (viii) | 95.3% | 97.0% | 98.5% | 99.1% |
| (ii), (iv), (vi)-(viii) | 89.9% | 92.8% | 96.6% | 97.8% |
| (i)-(iv), (vii), (viii) | 92.4% | 95.6% | 97.5% | 98.7% |
| (i), (ii), (vi), (v), (vii), (viii) | 94.8% | 97.4% | 98.2% | 99.2% |
| (i), (ii), (vi), (vi)-(viii) | 91.0% | 93.2% | 96.9% | 97.9% |
| (i)-(v), (vii), (viii) | 91.8% | 94.4% | 97.3% | 98.4% |
| (i), (ii), (iv)-(viii) | 92.1% | 93.3% | 97.4% | 97.9% |
| (i)-(viii) | 90.4% | 92.0% | 96.8% | 97.6% |

Seven (7) day averages
Features: (i) increased variability of weight; (ii) increased average number of daily urination events; (iii) similar duration of voiding phase of elimination events; (iv) decreased duration of finding/digging in litter; (v) decreased duration of covering; (vi) decreased duration of entirety of events; and/or (vii) decreased intensity of covering; (viii) decreased intensity of finding/digging.

In further detail, when a machine learning model may not be providing accurate enough predictions, modification of the model with an increased probability threshold and/or an overrule may be enough to improve the predictions. This can be helpful for cats, for example, that may exhibit a particular behavior that mimics some of the behaviors of cats experiencing renal health issues. As an example, a portion of mimicking behaviors in an otherwise healthy cat may result from some general unhappiness with a specific litter box. This may be corrected by adjusting the algorithm, e.g., increasing the number of eliminations, increasing the probability threshold, adding an overrule, etc. For example, in some instances, it was found that raising the number of eliminations in the machine learning model was found to increase the renal precision.

As mentioned, renal precision and healthy recall were identified as being helpful in making predictions regarding renal health. However, as stated, there may be other data that can provide insight into renal health, such as healthy precision and/or renal recall. In other examples herein, the use of multiple features at a 0.5 probability threshold (Table 3A), multiple features at a 0.7 probability threshold (Table 3B), and multiple features at a 0.7 probability threshold with an overrule applied (Table 3C) were compared for both precision, e.g., healthy precision and renal precision, and recall, e.g., healthy recall and renal recall. Additionally, weighted averages for precision data (both healthy and renal) and weighted averages for recall data (both healthy and renal) were also calculated. This data can be used to optimize or improve upon predictions of renal precision and healthy recall. In the data provided for this example, a 0.5 probability threshold resulted in acceptable renal precision prediction results at just above about 75% (See Table 3A). Furthermore, by increasing the probability threshold to 0.7, the renal precision was raised about 5% to at least about 80%. Furthermore, by adding an overrule (change in daily number or urinations), the renal precision was increased about an additional 7% to about 87%. This data is presented by way of example in Tables 3A-3C, as follows:

TABLE 3A
Predictions Using Multiple Features at 0.5 Probability Threshold

| Cat Condition | No. of Cats | No. of Events | Precision | Recall |
|---|---|---|---|---|
| Healthy | 33 | 1544 | 83.8% | 91.4% |
| Renal | 13 | 676 | 75.2% | 59.6% |
| Weighted Average | 46 | 2220 | 81.2% | 81.7% |

TABLE 3B
Predictions Using Multiple Features at 0.7 Probability Threshold

| Cat Condition | No. of Cats | No. of Events | Precision | Recall |
|---|---|---|---|---|
| Healthy | 33 | 1544 | 82.8% | 93.8% |
| Renal | 13 | 676 | 79.8% | 55.5% |
| Weighted Average | 46 | 2220 | 81.9% | 82.2% |

TABLE 3C
Predictions Using Multiple Features at
0.7 Probability Threshold and Overrule†

| Cat Condition | No. of Cats | No. of Events | Precision | Recall |
|---|---|---|---|---|
| Healthy | 33 | 1544 | 82.9% | 96.5% |
| Renal | 13 | 676 | 87.2% | 54.4% |
| Weighted Average | 46 | 2220 | 84.2% | 83.7% |

†Renal Prediction Heuristic Overrule

For additional clarity with respect to the data provided in Tables 3A-3C, renal precision is defined as the total number of true positives (true renal predictions) over total number of positive predictions (renal predictions); healthy recall is the total number of true positives (true healthy predictions) over total number of true positives (healthy cats); healthy precision is the total number of true positives (true healthy predictions) over total number of positive predictions (total healthy predictions); and renal recall is the total number of true positives (true renal predictions), over total number of true positives (renal cats). As mentioned, renal precision seems to provide the best data with respect to predicting the health state of the cat, but other types of predictive data can be used to increase prediction accuracy in some example.

### Predicting Other Health Conditions of Cats

The bulk of the data and disclosure presented herein relates to the prediction of renal health in cats. However, there are other diseases or conditions that cats may suffer from that could also benefit from combining the technology of the animal health monitoring systems including the smart litter box system with the development of machine learning models that are suitable for predicting any of a number of health conditions. Examples of other health conditions may include urinary tract disease, arthritis, hyperthyroidism, Feline idiopathic cystitis, chronic enteropathy, and/or diabetes, to name a few. For example, arthritis may be reasonably accurately predicted based on inputting features into a machine learning model (in combination with the use of data collection by the smart litter box system of the animal health monitoring system) that reliably predict the presence of the arthritis. Example features that may indicate this include decreased dig up duration, decreased total event duration, decreased dig up intensity, or decreased cover up intensity, to name a few. Urinary tract disease may be reasonably accurately predicted based on inputting features into a machine learning model that reliably predict the presence of the urinary tract disease, with features including increased total event duration, increased total visits, increased number of non-elimination visits, increased urination visits, or change (increased or decreased) in weight of output, to name a few. Hyperthyroidism may be reasonably accurately predicted based on inputting features into a machine learning model that reliably predict the presence of the hyperthyroidism, with features including increased dig up duration, increased weight of output, increased distance travelled in box, increased speed in box, or decrease in cat weight, to name a few. Other features that may lead to good predictions of hyperthyroidism may include differences in number of entries into the box and/or cover up phase duration. Feline idiopathic cystitis may be reasonably accurately predicted based on inputting features into a machine learning model that reliably predict the presence of the Feline idiopathic cystitis, with features including increased number of non-elimination events, increased defecation output duration, increased visits, change in weight of output, or decrease in total even duration, to name a few. Diabetes may be reasonably accurately predicted based on inputting features into a machine learning model that reliably predict the presence of the diabetes, with features including increased urination weight output, decreased defecation weight of output, decreased number of defecation events, or decreased cat weight, to name a few.

Table 4 below illustrates some of the raw data collected from a large population of cats that may be suitable to distinguish between healthy and compromised cats with multiple diseases or health conditions. These litter box behavioral patterns can be used in feature selection for building artificial intelligence for use with the smart litter box systems described herein in predicting the health state of cats.

TABLE 4
Litter Box Behavioral Patterns for Healthy and Compromised Cats

| | | Average Value | | Average | |
|---|---|---|---|---|---|
| | | for Health | | Value for | |
| | | Compromised | | Healthy | |
| Disease | Litter Box Behavior | Cats | | Cats | |
| Arthritis | Dig Up Duration | 13.3 | seconds | 17.0 | seconds |
| | Total Event Duration | 71.9 | seconds | 91.5 | seconds |
| | Dig Up Duration | 0.08 | second | 0.09 | second |
| | Cover Up Intensity † | 0.08 | | 0.19 | |
| Urinary Tract | Number of Visits | 2.9 | daily | 3.0 | daily |
| Disease | Total Event Duration | 100.6 | seconds | 71.9 | seconds |
| Hyperthyroidism | Dig Up Duration | 16.7 | seconds | 13.5 | seconds |
| Chronic | Non-elimination Events | 0.3 | daily | 0.1 | daily |
| Enteropathy | Defecation Output | 56.0 | grams | 40.6 | grams |
| | Total Event Duration | 73.9 | seconds | 91.5 | seconds |
| Diabetes | Urination Output | 156 | grams | 86.8 | grams |
| | Defecation Output | 17.5 | grams | 40.6 | grams |
| | Defecation Events | 0.4 | daily | 0.7 | daily |

† Intensity is based on peak density, which is the technical measurement of intensity.

Notably, when referring to the renal health of cats, renal precision was found to provide acceptable predictive values as described in detail throughout herein. However, for other diseases, such as those listed in Table 4 by way of example, there may be other data points that provide acceptable results, which may be even more predictive than precision. For example, for these and other diseases, other metrics can be used for making better predictions, such as healthy precision, healthy recall, healthy F1 scores, healthy support, compromised health precision, compromised health recall, compromised health F1 scores, compromised health support, etc., or combinations thereof. As an example, in some instances as it relates to hyperthyroidism, data collected related to healthy recall in one study was found to provide better predictive results than data collected related to hyperthyroid precision, though both may still be valuable metrics in making predictions. Thus, for each disease, data can be collected and models optimized using the principles described herein using the most predicative data for that particular disease. In further detail, predicting the compromised health of the cat can be carried out using any of these types of data (or combinations of types of data) with at least about 60% precision (or at least about 70% precision or at least about 75% precision) using at least a 0.5 probability threshold (starting at from about 0.5 to about 0.9 precision, e.g., at least 0.7 probability threshold) based on feature recognition of the at least one feature (or at least two features or at least three features, etc.).

It will be appreciated that all of the disclosed systems and methods and procedures described herein can be implemented using one or more computer programs, components, and/or program modules. These components may be provided as a series of computer instructions on any conventional computer readable medium or machine-readable medium, including volatile or non-volatile memory, such as RAM, ROM, flash memory, magnetic or optical disks, optical memory, or other storage media. The instructions may be provided as software or firmware and/or may be implemented in whole or in part in hardware components such as ASICs, FPGAs, DSPs, or any other similar devices. The instructions may be configured to be executed by one or more processors which, when executing the series of computer instructions, performs or facilitates the performance of all or part of the disclosed methods and procedures. As will be appreciated by one of skill in the art, the functionality of the program modules may be combined or distributed as desired in various aspects of the disclosure.

### Definitions

As used herein, “about,” “approximately” and “substantially” are understood to refer to numbers in a range of numerals, for example the range of −10% to +10% of the referenced number, −5% to +5% of the referenced number, −1% to +1% of the referenced number, or −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Thus, a disclosure of an embodiment using the term “comprising” includes a disclosure of embodiments “consisting essentially of” and “consisting of” the components identified.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive.

The term “average” may be used to describe a mean value or a median value. In many instances, the use of either of these values would be close enough so as to provide similar overall predictive results. In a minority of instances, however, the use of median values instead of mean values (or vice versa) may be more predictive, as may be determined by a skilled practitioner when building a machine-learning model in accordance with the present disclosure. With this in mind, the term “average” is used throughout the specification and typically relates to a mean value unless indicated otherwise. For example, the term “average” as used in the studies conducted and reported in Table 4 utilized median values (though it is believed that the use of mean values may generated very similar results).

As used herein, the “elimination” refers to the act of a cat voiding waste in the litter box, which includes both urination and defecation events. The document describes elimination events as those where the cat enters the litter box, finds a spot, performs the act of voiding (either urination or defecation), and then covers the waste before exiting. Elimination events are specifically tracked and analyzed using load sensors in a smart litter box system to monitor health-related behaviors. Similarly, as used herein, the term “urination” is meant as a type of elimination event where the cat voids urine in the litter box. The document distinguishes urination events by their typical duration (shorter than defecation events but longer than non-elimination events) and by the increase in weight detected by the load sensors after the event, as urination usually results in a larger weight increase than defecation. Urination frequency and characteristics are key features used in machine learning models to predict renal health and other conditions. Additionally, as used herein, “defecation” refers to another type of elimination event, specifically referring to the act of a cat voiding feces in the litter box. Defecation events are characterized by their duration (typically longer than urination events), and the weight change detected by the load sensors is generally less than that of urination. The frequency and characteristics of defecation events are also tracked as features for health prediction.

As used herein, the term “litter” means any substance that can absorb animal urine and/or decrease odor from animal urine and/or feces. A “clumping litter” forms aggregates in the presence of moisture, where the aggregates are distinct from the other litter in the litter box. A “clumping agent” binds adjacent particles when wetted. A “non-clumping litter” does not form distinct aggregates.

The term “litter box” means any apparatus that can hold pet litter, for example a container with a bottom wall and one or more side walls, and/or any apparatus configured for litter to be positioned thereon, for example a mat or a grate. As a non-limiting example, a litter box may be a rectangular box having side walls that have a height of at least about six inches. A litter box may rest on top of a platform equipped with load cells, or may be integrated with (or integrated as part of) a platform equipped with load cells, for example. Thus, the combination of a litter box (integrated with a platform or resting on top of a platform) combined with a plurality of load cells may be referred to herein as a “smart litter box system.”

Although the present disclosure has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present disclosure can be practiced otherwise than specifically described without departing from the scope and spirit of the present disclosure. Thus, aspects of the present disclosure should be considered in all respects as illustrative and not restrictive. It will be evident to the annotator skilled in the art to freely combine several or all of the aspects discussed here as deemed suitable for a specific application of the disclosure. Throughout this disclosure, terms like “advantageous”, “exemplary” or “preferred” indicate elements or dimensions which are particularly suitable (but not essential) to the disclosure or an embodiment thereof, and may be modified wherever deemed suitable by the skilled annotator, except where expressly required. Accordingly, the scope of the present disclosure should be determined not by the embodiments illustrated, but by the appended claims and their equivalents.

What is claimed is:

1. A method of predicting renal health of a cat, under the control of at least one processor, comprising:
obtaining load data over time from interactions of a cat with a smart litter box system, wherein the smart litter box system is associated with or includes a plurality of load sensors that are separated from one another and independently receive changing pressure inputs from the smart litter box system resulting from the interactions;
using the load data in conjunction with a machine learning model, the machine learning model including feature recognition of at least one feature based on the load data, wherein the at least one feature indicates compromised renal health compared to benchmark values established from a population of healthy cats, and wherein the at least one feature includes:
increased number of daily elimination events, or
multiple features selected from increased variability of weight, increased number of daily urination events, similar duration of voiding phase of elimination events, decreased duration of finding/digging in litter, decreased duration of covering, decreased duration of entirety of events, decreased intensity of covering, decreased intensity of finding/digging, decreased number of defecation events, decreased weight of elimination, or increased number of smart litter box system visits; and
predicting the compromised renal health of the cat is with at least about 75% renal precision using at least a 0.5 probability threshold based on the feature recognition of the at least one feature.

2. The method of claim 1, wherein the at least one feature includes the increased number of daily elimination events.

3. The method of claim 2, wherein the at least one feature further includes one or more of increased variability of weight, similar duration of voiding phase of elimination events, decreased duration of finding/digging in litter, decreased duration of covering, decreased duration of entirety of events, decreased intensity of covering, decreased intensity of finding/digging, decreased number of defecation events, decreased weight of elimination, or increased number of smart litter box system visits.

4. The method of claim 3, wherein predicting the renal health of the cat is with at least about 80% renal precision.

5. The method of claim 2, wherein the at least one feature further includes two or more of increased variability of weight, similar duration of voiding phase of elimination events, decreased duration of finding/digging in litter, decreased duration of covering, decreased duration of entirety of events, decreased intensity of covering, decreased intensity of finding/digging, decreased number of defecation events, decreased weight of elimination, or increased number of smart litter box system visits.

6. The method of claim 5, wherein predicting the renal health of the cat is with at least about 85% renal precision.

7. The method of claim 1, wherein the probability threshold is at least about 0.7.

8. The method of claim 7, wherein predicting the renal health of the cat is with at least about 80% renal precision.

9. The method of claim 1, wherein predicting the renal health of the cat includes applying an overrule to increase the renal precision.

10. The method of claim 9, wherein the overrule is a renal precision heuristic overrule based on a change in number of daily urinations being required for a prediction of a renal health compromised cat.

11. The method of claim 1, wherein obtaining the load data over time includes calculating a cumulative load over time based on the load data obtained independently by the plurality of load sensors.

12. An animal health monitoring system, comprising:
a smart litter box system associated with or including a plurality of load sensors to obtain load data, wherein individual load sensors of the plurality of load sensors are separated from one another and independently receive pressure inputs as a result of cat interaction with the smart litter box system and litter contained therein;
a data communicator configured to communicate the load data from the plurality of load sensor;
a processor; and
a memory storing instructions that, when executed by the processor:
receives the load data from the data communicator,
uses the load data in conjunction with a machine learning model that includes feature recognition of at least one feature based on the load data, wherein the at least one feature indicates compromised health compared to benchmark values established from a population of healthy cats, and
predicts the compromised health of the cat is with at least about 60% precision using at least a 0.5 probability threshold based on feature recognition of the at least one feature.

13. The animal health monitoring system of claim 12, wherein the at least one feature indicates compromised renal health compared to benchmark values established from a population, and wherein the at least one feature includes:
increased number of daily elimination events, or
multiple features selected from increased variability of weight, increased number of daily urination events, similar duration of voiding phase of elimination events, decreased duration of finding/digging in litter, decreased duration of covering, decreased duration of entirety of events, decreased intensity of covering, decreased intensity of finding/digging, decreased number of defecation events, decreased weight of elimination, or increased number of smart litter box system visits.

14. A method of predicting a health state of a cat, under the control of at least one processor, comprising:
obtaining load data over time from interactions of a cat with a smart litter box system, wherein the smart litter box system is associated with or includes a plurality of load sensors that are separated from one another and independently receive changing pressure inputs from the smart litter box system resulting from the interactions;
using the load data in conjunction with a machine learning model, the machine learning model including feature recognition of at least one feature based on the load data, wherein the at least one feature indicates compromised health compared to benchmark values established from a population of healthy cats; and
predicting the compromised health of the cat is with at least about 60% precision using at least a 0.5 probability threshold based on feature recognition of the at least one feature.

15. The method of claim 14, wherein predicting the unhealthy state includes predicting the presence of a health condition selected from:
urinary tract disease with the at least one feature used for predicting the urinary tract disease includes increased total event duration, increased total visits, increased number of non-elimination visits, increased urination visits, or change in weight of output;
arthritis with the at least one feature used for predicting the arthritis includes decreased dig up duration, decreased total event duration, decreased dig up intensity, or decreased cover up intensity;
hyperthyroidism with the at least one feature used for predicting the hyperthyroidism includes increased dig up duration, increased weight of output, increased distance travelled in box, increased speed in box, or decrease in cat weight;
feline idiopathic cystitis with the at least one feature used for predicting the Feline idiopathic cystitis includes increased number of non-elimination events, increased defecation output duration, increased visits, change in weight of output, or decrease in total even duration; or
diabetes with the at least one feature used for predicting the diabetes includes increased urination weight output, decreased defecation weight of output, decreased number of defecation events, or decreased cat weight.
